(19) United States
(12) Patent Application Publication
(10) Pub. No.: US 2013/0338240 A1
(43) Pub. Date: Dec. 19, 2013

(54) USE OF A VISCOELASTIC FLUID FOR PRODUCING A MEDICINAL PRODUCT FOR SURGICALLY TREATING THE EYE

(71) Applicant: Oliver Findl, Wien (AT)

(72) Inventor: Oliver Findl, Wien (AT)

(73) Assignee: CROMA-PHARMA GMBH, Leobendorf (AT)

(21) Appl. No.: 14/002,438

(22) PCT Filed: Mar. 5, 2012
(86) PCT No.: PCT/EP2012/053710

§ 371 Date: Aug. 30, 2013

(30) Foreign Application Priority Data
Mar. 3, 2011 (AT) A 288/2011

(51) Int. Cl. A61K 47/38

(52) U.S. Cl. CPC A61K 47/38
USPC 514/781

(57) ABSTRACT

The invention relates to the use of an aqueous viscoelastic fluid for producing a medicinal product for the surgical treatment of the eye, which fluid produces an optical magnifying effect of the lens and the pupil upon application onto the surface of the eye from a single-dose receptacle.

The viscoelastic fluid comprises at least one viscosity-increasing, physiologically acceptable polymer selected from the group consisting of hydroxypropylmethyl cellulose, carboxymethyl cellulose, hydroxyethyl cellulose, hyaluronic acid, sodium alginate, hydroxylpropyl guar polyvinylpyrrolidone, polyvinyl alcohol, polymethacrylic acid (carbomer), polyoxyethylene polyoxypropylene copolymer (poloxamer) and polyethylene glycol, at a concentration of 0.01-30%.

The invention relates to the use of a viscoelastic fluid for producing a medicinal product for the surgical treatment of the eye, which fluid produces a magnifying effect of the lens and the pupil upon application onto the surface of the eye.

The viscoelastic fluid serves for protecting the cornea from desiccation as well as from damages to the epithelium during eye surgery such as cataract surgery, glaucoma surgery, removal of foreign objects or surgery of the rear area of the eye (posterior segment surgery such as, e.g., vitrectomy, trabeculectomy).

During surgery, the eye is usually moistened with a saline solution at regular intervals in order to prevent the cornea from desiccation. However, this process interrupts the activity of the surgeon, impairs the surgical progress and destroys the homeostasis of the tear film. In further consequence, important components of the tear film such as, e.g., anti-inflammatory enzymes, lipids, mucopolysaccharides are thereby washed out.

It is known to use a viscoelastic fluid for moistening the eye prior to the surgical treatment of a cataract, which fluid efficiently protects the cornea from desiccation during the surgery. In doing so, the fluid is squeezed from a syringe and, if necessary for an optimum distribution, is spread on the cornea by means of a spatula or a microsponge.

It is the object of the invention to provide a medicinal product with a viscoelastic fluid, which should produce a magnifying effect of the lens and the pupil upon application onto the eye.

It has been shown that a viscoelastic fluid having the following composition produces a magnifying effect of the lens and the pupil to the extent of 10-15%:

The formulation comprises at least one viscosity-increasing, physiologically acceptable polymer which is known to moisten the surface of the eye, such as, for example, hydroxypropylmethyl cellulose, carboxymethyl cellulose, hydroxyethyl cellulose, hyaluronic acid, sodium alginate, hydroxylpropyl guar polyvinylpyrrolidone, polyvinyl alcohol, polymethacrylic acid (carbomer), polyoxyethylene polyoxypropylene copolymer (poloxamer), polyethylene glycol,
at a concentration of 0.01-30%
or a combination of two or several of said polymers.

The composition according to the invention is applied at the eye. Therefore, the composition preferably has a pH value ranging from 6 to 8.5, preferably from 6.5 to 8, even more preferably from 6.8 to 7.6.

For administration of compositions at the eye, it is furthermore advantageous if said compositions have an osmolarity comparable to that of the tear fluid. Therefore, the osmolarity of the composition according to the invention preferably ranges from 200 to 400 mosmol/1, even more preferably from 280 to 330 mosmol/1.

The auxiliary agents required therefore, such as, e.g., buffer salts, stabilizers, auxiliary agents for adjusting the desired osmolarity and auxiliary agents for increasing the tolerance, depend on the respective formulation and are sufficiently known to a person skilled in the art.

### Example of a Viscoelastic Fluid:

### Cornea Protect Composition:

water for injection purposes
sodium hydroxide
lactic acid 90%
sodium chloride
potassium chloride
calcium chloride×2H₂O
hydroxypropylmethyl cellulose
Filling volume: 2 ml
pH value: 6.8-7.6
Osmolarity: 265-330 mOsmol/kg

A concrete formulation according to the invention is as follows, wherein the amounts of the indicated substances refer to 1 ml of water for injection purposes:
sodium hydroxide: 1.15 mg
lactic acid 90%: 2.40 mg
sodium chloride: 6.00 mg
potassium chloride: 0.40 mg
calcium chloride×2H2O: 0.27 mg
hydroxypropylmethyl cellulose: 22.00 mg

Instead of hydroxypropylmethyl cellulose, hyaluronic acid may preferably also be present at an amount of 0.01-10%, in particular of 15.4 mg/ml. In this case, the following furthermore may be present:
sodium chloride: 8.15 mg/ml
di-sodium hydrogen phosphate dodecahydrate: 0.70 mg/ml
sodium dihydrogen phosphate dihydrate: 0.056 mg/ml

The pH value preferably is in the range from 6.8 to 7.6, and the osmolarity is in the range of between 280 and 330.

In the following example, the optical magnifying effect of the lens and the pupil is illustrated by way of an artificial eye.

### EXAMPLE

2 ml of the above described viscoelastic fluid weres applied onto a model eye. In comparison to an untreated model eye, the optical magnifying effect was about 10%.

The viscoelastic fluid may be contained in a receptacle shrink-wrapped in a protective cover, which receptacle is used as a medicinal product for eye surgery. Furthermore, the invention relates to the receptacle shrink-wrapped in the protective cover.

The receptacle is designed such that the fluid can be taken out from the receptacle via a predetermined breaking point, the production method thereby being characterized by a combination of the features that
the viscoelastic fluid used according to the invention is filled into the receptacle, whereupon said receptacle is closed,
the closed receptacle is shrink-wrapped in a protective cover, whereupon
the shrink-wrapped receptacle including the protective cover is subjected to thermal sterilization.

After the receptacle has been shrink-wrapped in the protective cover, internal and external sterility of the product is ensured by the terminal sterilization of the product. A further advantage of the method according to the invention is that no preservative are to be added to the viscoelastic fluid.

The receptacle produced according to the invention guarantees higher convenience for the surgeon during its use, as well as more safety for the patient.

A preferred embodiment of the method according to the invention consists in that the receptacle is a single-dose receptacle.

The receptacle or single-dose receptacle, respectively, is preferably made from polypropylene or mixtures of polyethylene or polypropylene with copolymers of ethylene and propylene or from a laminate.

The protective cover preferably consists of a sterilizable medicinal paper and a composite film (e.g., Medipeel® Pouch from Sengewald) or Tyvek® material (manufacturer DuPont).

The thermal sterilization may be performed at a temperature between 80 and 140° C.

The invention furthermore relates to the receptacle which can be produced according to the method of the invention and is shrink-wrapped in a protective cover as such.

The single-dose receptacles preferably consist of pharmaceutical grade polypropylene (PP). The polypropylene raw material which is preferably used for the production of the single-dose receptacles has the following properties:

Melting point (determined according to ISO 3146): 100° C.-260° C.

Vicat softening temperature (10N, 50° C. per hour; determined according to ISO 306): 80° C.-240° C.;

Melt flow index (230° C. /2.16 kg; determined according to ISO 1133): 0.1 g/10 min-50 g/10 min;

Tensile strain at yield (50 mm/min; determined according to ISO 527-2): 1%-30%;

Charpy notched impact strength (at 23° C.; determined according to ISO 179): 1 kj/m² -20 kj/m²

The protective cover for the single-dose receptacle is preferably made up of a sterilizable medicinal paper and a special composite film, with one side being transparent (e.g. Medipeel®Pouch from Sengewald), and ensures external sterility of the single-dose receptacle.

### Description of the Sterilization

Due to the specific packaging of the viscoelastic fluid in a single-dose receptacle and a overlying protective cover, internal and external sterility of the product is thus ensured in a single—terminal—sterilization step. The preferred type of sterilization is the physical sterilization by heat in a temperature range from 80° C. to 140° C., for example, by hot-water sprinkling, saturated-steam sterilization or sterilization with a steam-air-mixture.

Surprisingly, it has been shown that the terminal sterilization with ionizing rays is inappropriate in the present case, since it results in an uncontrolled degradation of the viscosity-increasing polymers in the fluid and the product no longer displays the necessary viscoelastic properties after sterilization. All other sterilization methods have the drawback that the sterilization cannot be performed as the terminal sterilization in the final receptacle, but two sterilization steps for internal and external sterility would be required.

1. Use of an aqueous viscoelastic fluid for producing a medicinal product for the surgical treatment of the eye, which fluid produces an optical magnifying effect of the lens and the pupil upon application onto the surface of the eye from a single-dose receptacle, characterized in that the viscoelastic fluid
a) comprises at least one viscosity-increasing, physiologically acceptable polymer selected from the group consisting of hydroxypropylmethyl cellulose, carboxymethyl cellulose, hydroxyethyl cellulose, hyaluronic acid, sodium alginate, hydroxylpropyl guar polyvinylpyrrolidone, polyvinyl alcohol, polymethacrylic acid (carbomer), polyoxyethylene polyoxypropylene copolymer (poloxamer) and polyethylene glycol, at a concentration of 0.01-30%,
b) has a pH value ranging from 6 to 8.5, preferably from 6.5 to 8, even more preferably from 6.8 to 7.6, and
c) has an osmolarity ranging from 200 to 400 mosmol/1, preferably from 280 to 330 mosmol/1.

2. A method for producing an optical magnifying effect of the lens and the pupil of an eye of a patient which comprises applying a viscoelastic fluid onto the surface of the eye of a patient in need thereof, wherein the viscoelastic fluid is contained in a single-dose receptacle, and wherein said viscoelastic fluid
a) comprises at least one viscosity-increasing, physiologically acceptable polymer selected from the group consisting of hydroxypropylmethyl cellulose, carboxymethyl cellulose, hydroxyethyl cellulose, hyaluronic acid, sodium alginate, hydroxylpropyl guar polyvinylpyrrolidone, polyvinyl alcohol, polymethacrylic acid (carbomer), polyoxyethylene polyoxypropylene copolymer (poloxamer) and polyethylene glycol, at a concentration of 0.01-30%,
b) has a pH value ranging from 6 to 8.5, and
c) has an osmolarity ranging from 200 to 400 mosmol/1.

3. The method according to claim 2, wherein said pH ranges from 6.5 to 8.

4. The method according to claim 2, wherein said pH ranges from 6.8-7.6.

5. The method according to claim 2, wherein said osmolarity ranges from 280 to 330 mosmol/1.

6. The method according to claim 4, wherein said osmolarity ranges from 280 to 330 mosmol/1.
